(19) United States
(12) Patent Application Publication
(10) Pub. No.: US 2015/0274374 A1
(43) Pub. Date: Oct. 1, 2015

(54) STERILE ELECTRONICS BAG AND STERILE CLOSURE MECHANISM

(71) Applicant: Whitney Products, Inc., Niles, IL (US)

(72) Inventors: Steven G. Whitney, Glenview, IL (US); Allison Bedell, Winnetka, IL (US)

(73) Assignee: Whitney Products, Inc.

(21) Appl. No.: 14/739,130

(22) Filed: Jun. 15, 2015

Related U.S. Application Data
(62) Division of application No. 13/179,370, filed on Jul. 8, 2011.
(60) Provisional application No. 61/363,203, filed on Jul. 9, 2010.
(60) Provisional application No. 61/411,906, filed on Nov. 9, 2010.

(51) Int. Cl. B65D 33/04; B65D 33/00; B65B 7/02; A61B 19/00; B65B 5/04; A45C 11/38; B65D 33/16

(52) U.S. Cl. CPC B65D 33/04; A45C 11/38; B65D 33/004; B65D 33/1616; A61B 19/38; B65B 5/045; B65B 7/02

(57) ABSTRACT

A sterile bag for containing non-sterile objects includes: a body; a sealed first end; a second end forming a cuff and an opening to an internal portion of the bag when in a first position; a visible demarcation located circumferentially along the cuff dividing the bag into two sides; a closure mechanism located along the cuff on the proximal side of the visible demarcation such that when the second end is in a second position there is no cuff and the closure mechanism can be used to seal the bag with the visible demarcation sealed within the bag. In use, a first user with scrubbed hands may touch the bag only on one side of the visible demarcation and a second user with non-scrubbed hands may touch the bag only on the other side of the visible demarcation.

### CROSS-REFERENCE TO RELATED APPLICATIONS

This application claims the benefit of priority to and is a divisional of U.S. patent application Ser. No. 13/179,370 filed Jul. 8, 2011. In addition, this application incorporates by reference and claims the benefit of priority to U.S. Provisional Patent Application No. 61/363,203 filed Jul. 9, 2010, and U.S. Provisional Patent Application No. 61/411,906 filed Nov. 9, 2010.

### BACKGROUND OF THE INVENTION

The present subject matter relates generally to a sterile cover for electronics and closure mechanism for the same. More specifically, the present invention relates to a sterile camera cover having means for isolating the lens from the remainder of the interior of the cover including a sterile closure mechanism.

There are times during surgery, as well as in other clean or sterile environments and applications, when it is advantageous to make use of non-sterile or difficult-to-sterilize items and materials. For example, one may wish to use cameras, computers, cell phones, and other electronic devices in a sterile environment. Medical personnel working in a sterile environment such as an operating room, need to be able to take photographs and video of patients, surgical procedures, etc. during the procedure. In these cases, it would be advantageous to have a sterile barrier into which a person, whose hands are not scrubbed/sterile, can place the non-sterile item/material, without compromising the sterility of the exterior of the barrier.

At present, “scrubbed” personnel must either rely on other personnel with non-scrubbed hands to take the photos or manipulate non-sterile devices, or must handle the non-sterile camera or device themselves, requiring clean-up before handling the camera, and re-scrubbing and re-gloving prior to continuing the medical procedure.

With respect to maintaining the sterility of the bag containing the non-sterile device or materials, currently, x-ray cassettes are employed using a large plastic bag with a fold-over opening, similar to a classic sandwich bag. These bags fail to meet the objectives of the invention described herein in that the x-ray bags are limited in that: (1) it is difficult and awkward for the user(s) to handle the non-sterile cassette and close the bag without compromising the sterility of the barrier; (2) it is difficult to identify which areas of the bag have uncompromised sterility and are thereby free to be touched/handled by the user whose hands are scrubbed/sterile; and (3) it is difficult to secure the bag closed to avoid transfer of contamination from the interior to the exterior.

Accordingly, there is a need for a cover to be used in conjunction with a camera (still or video, film or digital) or other item, to provide a sterile outside surface for handling and controlling the camera, and preventing body fluids from coming into contact with the non-sterile camera or item inside the cover.

### SUMMARY OF THE INVENTION

The present invention provides a clear plastic bag as a sterile cover for a camera for use in surgery or other sterile environments. A lens window is provided with sufficient clarity to allow photography through it. The lens window may be the wall of the bag itself made from sufficiently optically clear flexible material. Alternatively, the lens window may be a discrete flexible or rigid window attached across a hole in the wall of the bag.

The lens window may be held directly against the bezel of the lens by mechanical means. For example, a double-sticky ring of material may be used to adhere the lens bezel to the lens window area. The lens ring may be selected from amongst a plurality of concentric rings die-cut from the same piece of material as a means to provide a variety of lens rings for selection by the user. Use of die-cut tabs on the concentric rings may allow easier removal and positioning of the lens ring. The bezel rings are merely one example of an adhesive material used to secure the bezel to the lens window, the rings may instead be tabs, strips, bars, etc. Alternatively, a mechanical clip, screw-on lens filter, adjustable ring, or band may be used to attach the lens bezel to the lens window area.

In addition, the present invention provides a bag including a sterile outer surface and a non-sterile inner surface without compromising the sterility of the outer surface or of any other sterile objects involved. The bag may be utilized such that a person with scrubbed/sterile hands may handle the exterior of the bag and a person with non-scrubbed hands may handle the non-sterile contents, allowing for the non-sterile materials to be enclosed within a sterile exterior, which may be handled and used within a sterile environment. To this end, the present invention may include a protective cuff, a visible demarcation between the sterile and non-sterile zones and a secure closure.

In one embodiment, a cuff of material may be provided to assist in keeping a scrubbed person's hands out of contact with camera as it is placed in the bag. Double-sticky tape may be used as part of a closure for the bag. An oversized (wider) release liner may be used for the closure tape to allow easier manipulation and removal by gloved hands. Alternatively, an extruded “zipper-style” closure may be provided for the bag.

It is contemplated that the sterile bag and method for placing a non-sterile object within a bag may be independently executed without the need for a lens window. A summary of such an embodiment is provided as follows.

In use, a sterile bag may be provided in a standard sterilization package such as a pouch, tray or other sterilization package. A person with scrubbed hands may remove the bag, unfold it, and place his or her hands beneath the protective cuff. The protective cuff provides a place for the person with scrubbed hands to hold the bag open while the non-sterile contents are inserted. The person with scrubbed hands can then support and manipulate the bag from under the cuff. The layer of sterile material forming the protective cuff covers the hands, adding extra assurance that the hands will not come in contact with non-sterile contents as they are inserted.

The bag may include a visible demarcation, such as, for example, a solid line or other visible indication, located circumferentially around the cuff. The demarcation visibly separates the designated non-sterile part from the sterile part. Both parties (i.e., the scrubbed user and the non-scrubbed user) must understand which side of the demarcation they are permitted to touch. The person with non-scrubbed hands may only touch the interior surfaces of the bag and the exterior of the cuff distal to the visible demarcation. The person with scrubbed hands may only touch the exterior of the bag and the exterior of the cuff proximal to the visible demarcation. The visible demarcation serves the purpose of removing any uncertainty about which surfaces of the bag are being maintained sterile and which are not.

After all of the non-sterile contents have been added to the bag, the cuff may be involuted by the person whose hands are scrubbed, by manipulating the cuff from below, until the visible demarcation is inside the bag. The bag may be sealed above/outside the demarcation by a secure closure. Numerous closure mechanisms may be employed. For example, potential closures may include, but are not limited to, zippers, tape, rubber bands, and collars. The closure mechanism may be located inside or outside of the bag, but should be positioned in the sterile area to assure that the non-sterile contents are securely contained, enabling the secured bag to be handled casually by sterile hands.

Depending on the configuration of the sterile bag and closure mechanism, there may be excess material near the opening of the bag remaining within the sterile zone that will no longer be needed once the non-sterile materials are located within the bag. In such embodiments, the excess material of the bag located outside of the closure mechanism may be perforated for removal after the bag is sealed. For example, the excess material necessary to form the protective cuff, but unneeded after the bag is closed, may be eliminated in this manner. The perforation should be located above/outside closure, in the sterile area of the bag.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 illustrates an example of a sterile bag.

FIG. 2 illustrates an example of the sterile bag from FIG. 1 shown with a user holding the bag open.

FIG. 3 illustrates an example of a sterile bag with a band closure mechanism.

FIGS. 4A and 4B illustrate an example of a sterile bag with a double-stick adhesive closure mechanism.

FIGS. 5A and 5B illustrates an example of a sterile bag with a zipper closure mechanism.

FIGS. 6A and 6B illustrates an example of a sterile bag with another adhesive closure mechanism.

FIG. 7 is an example of a sterile bag with a perforated removable section.

FIG. 8 is an example of a sterile bag with a lens window.

FIG. 9 is an example of a camera and adhesive bezel ring.

FIG. 10 is an example of a plurality of bezel rings.

FIG. 11 is an example of a plurality of bezel rings with tabs.

### DESCRIPTION OF THE PREFERRED EMBODIMENTS

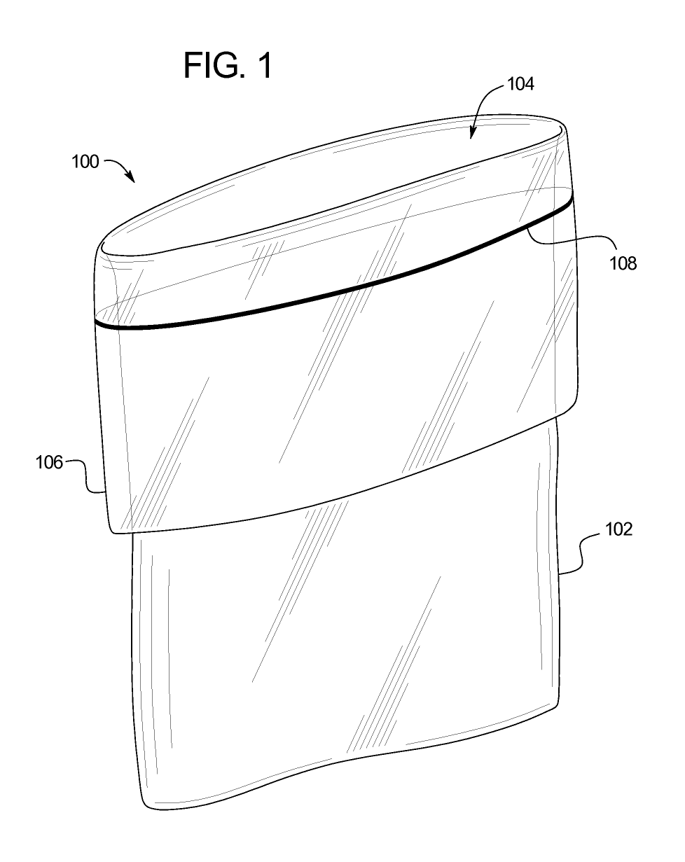

As shown in FIG. 1, the present subject matter provides an optically clear plastic bag 100, the outside surface 102 of which is sterile and can be handled by scrubbed personnel, and the inside surface 104 of which can contain non-sterile objects, such as a camera or other electronics, safe from contact with body fluids. As described further herein, the bag 100 may include a cuff 106 that includes a visible demarcation 108 to delineate the bag 100 into distal and proximal sides of the visible demarcation 108.

As used herein, the terms proximal and distal refer to portions of the bag 100 with reference to the distance from the outside surface 102 at the sealed bottom portion of the bag 100. The most proximal portion of the bag 100 is at the outside surface 102 at the sealed bottom end of the bag 100. As one moves along the surface of the bag 100, starting on the outside surface 102 of the body of the bag 100, towards the open mouth, and then into the inside surface 104 of the bag 100, one moves more distal along the bag 100. Accordingly, the most distal portion of the bag 100 is at the sealed bottom of the bag 100 on the inside surface 104. As described further herein, the visible demarcation 108 divides the bag 100 into two main sections, the more proximal section is intended to always remain sterile and the more distal section is intended to potentially contact non-sterile objects and surfaces, such as an object inserted into the bag 100 or the skin of a non-scrubbed user inserting an object into the bag 100.

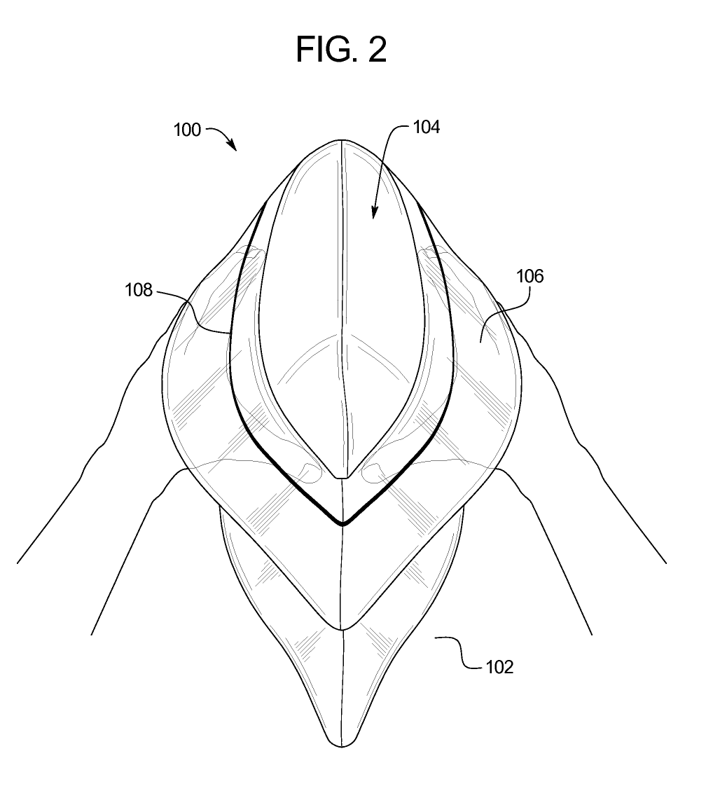

The bag 100 may be provided sterile in a peel-open package (or other sterile mechanism), and a scrubbed person, contacting only the outside surfaces of the bag, may hold the bag 100 open for a non-scrubbed person to place a non-sterile object inside the bag 100. This is similar to the procedure commonly used for wrapping x-ray plates and other non-sterile items that are used in the operating field. FIG. 2 illustrates an example of a scrubbed user holding the bag 100, with the user's hands protected under the cuff 106 on the proximal side of the visible demarcation 108. As shown in FIG. 2, the cuff 106 allows the scrubbed person to more easily hold the bag 100 open without contacting any non-sterile object that may be placed into the bag 100.

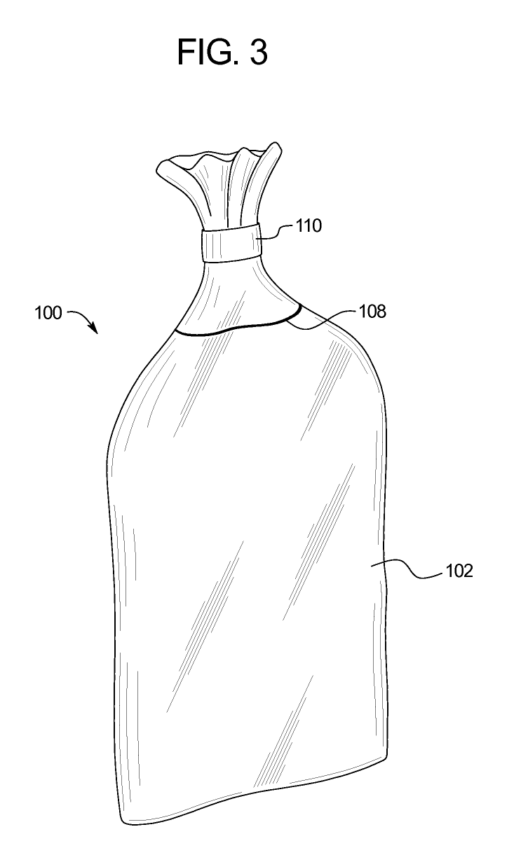

The bag 100 may include a closure mechanism. It is contemplated that the closure mechanism may be embodied in any of a number of forms. For example, in the embodiment shown in FIG. 3, the closure mechanism may be something as simple as a band 110 which seals the bag 100. The band 110 may be an elastomeric material, an adhesive material, etc.

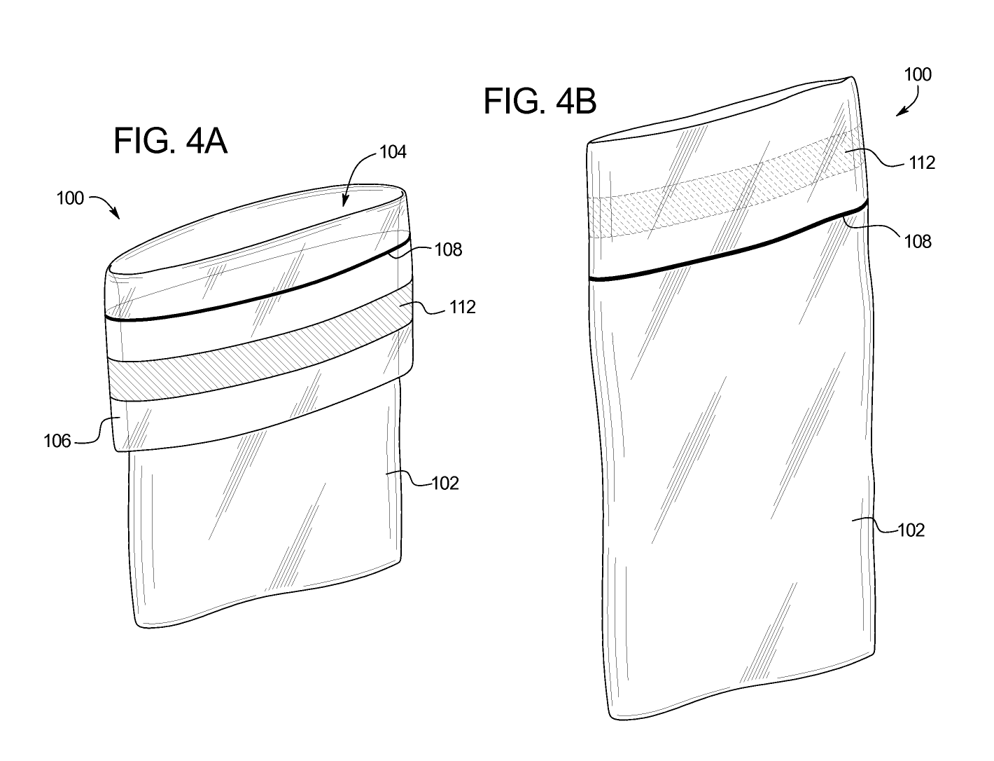

In another example shown in FIGS. 4A and 4B, the closure mechanism may be a double-stick adhesive material closure 112 that seals the bag 100 when the cuff 106 is involuted. If using an adhesive material closure 112, it may be advantageous to include a release liner that extends beyond the edge of the adhesive material, to allow the scrubbed (and likely gloved) user to more easily grasp and remove the release liner without contacting the adhesive material.

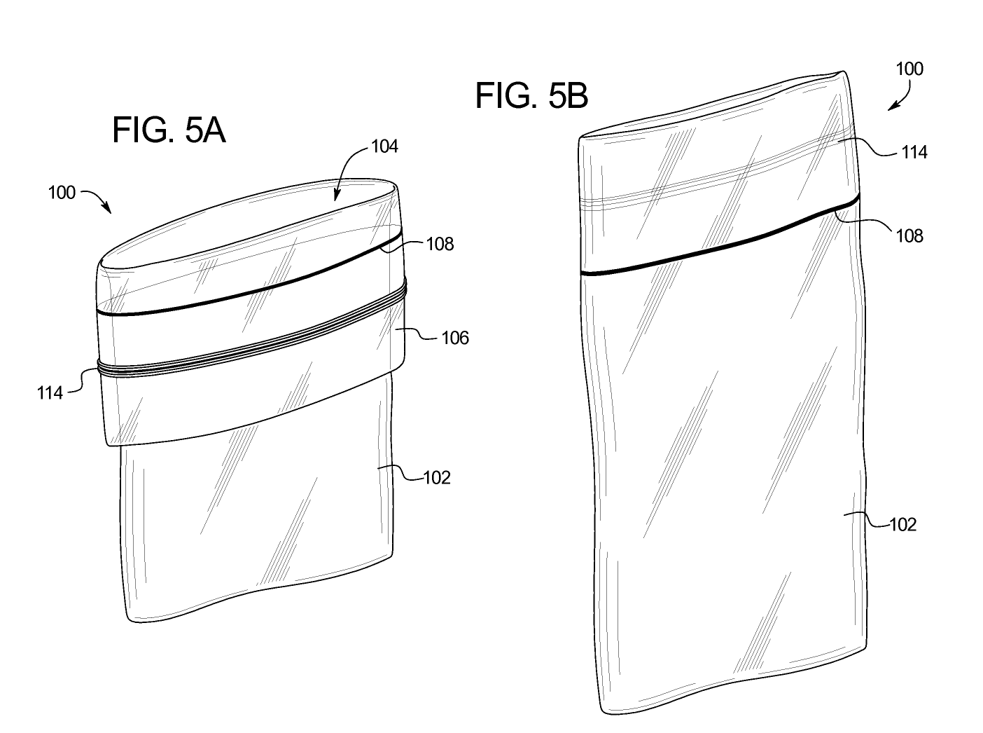

In a further example shown in FIGS. 5A and 5B, the closure mechanism may be embodied in a zipper mechanism 114. The zipper mechanism 114 may be, for example, a plastic zipper such as those commonly used on plastic bags sold by S.C. Johnson & Son under the trademark Ziploc.

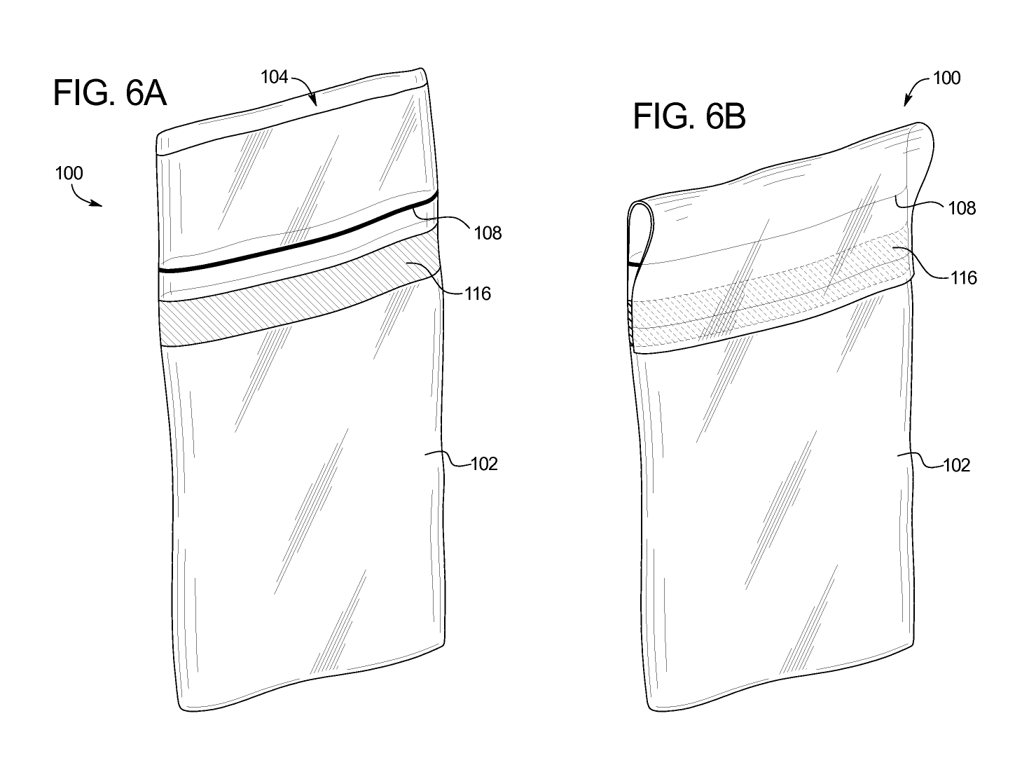

In yet another example shown in FIGS. 6A and 6B, the closure mechanism may be embodied in another version of an adhesive material closure 116. In this example, the adhesive material closure 116 is provided on one side of the bag 100, such that the involuted cuff 106 may be folded over one or more times to contact the adhesive material closure 116 to seal the bag 100.

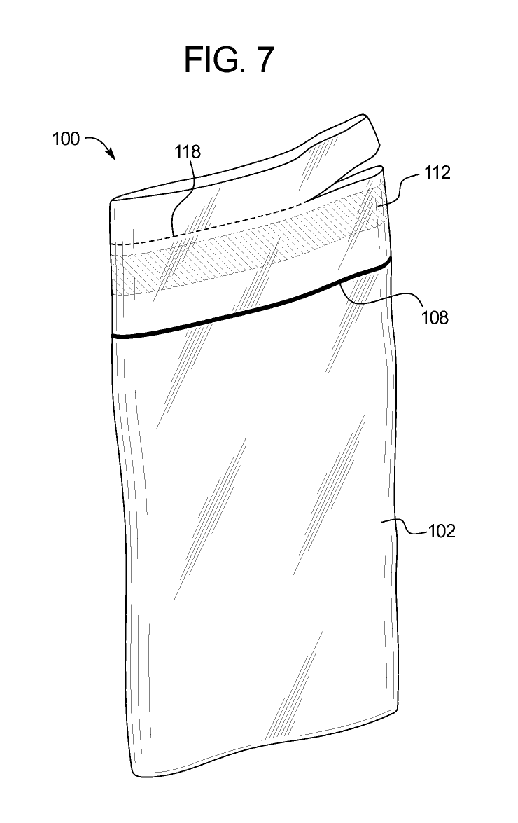

Depending on the configuration of the bag 100 and closure mechanism, there may be excess material remaining that is no longer needed after the non-sterile materials are sealed within the bag 100. In such embodiments, the excess material located proximal to the closure mechanism may be perforated 118 for removal after the bag 100 is sealed, as shown in FIG. 7. For example, the excess material may be necessary to form the protective cuff 106, but may be unneeded after the bag 100 is sealed and may be eliminated in this manner. The perforation 118 should be located distal to the closure mechanism, while still within in the sterile area of the bag 100.

The walls of the bag 100 may be sufficiently transparent or translucent to enable a user to see and manipulate any object sealed within the bag 100, for example to operate the controls and displays of a camera other electronic device sealed within the bag 100.

The material used to form the walls of the bag 100 may be sufficiently tough to prevent damage during normal use, but sufficiently flexible to allow manipulation of the camera or other object through the surface of the bag 100.

As taught herein, it may be advantageous to provide a sterile bag 100 that includes a visible demarcation 108 between the sterile and non-sterile portions of the bag 100. The visible demarcation 108 may enable users to quickly visually identify which portion of the bag 100 they may handle (e.g., users with scrubbed hands may handle the bag 100 on the proximal side of the visible demarcation 108 and users with non-scrubbed hands may handle the bag 100 on the distal side of the visible demarcation 108). As used herein, the proximal side of the visible demarcation 108 is the sterile portion of the bag 100 and distal side of the visible demarcation 108 is the portion of the bag 100 that may contact non-sterile materials or be touched by the unscrubbed user.

The following is an example of the use of an embodiment of the bag 100. In use, the bag 100 may be provided in a standard sterilization package such as a pouch, tray or other package. A person with scrubbed hands may remove the bag 100, unfold it, and place his or her hands below the protective cuff 106. The protective cuff 106 provides a place for the person with scrubbed hands to hold the bag 100 open while the non-sterile contents are inserted. The person with scrubbed hands can then support and manipulate the bag 100 and its contents through the wall of the bag from under the protective cuff 106. The protective cuff 106 covers the hands, adding extra assurance that the hands will not come in contact with non-sterile contents as they are inserted into the bag 100. The size of the protective cuff 106 may vary depending on the application, but it is contemplated that the protective cuff 106 will be adequately sized to cover the scrubbed hands of the user and adequately protect the user from being in contact with any non-sterile items, such as, for example the item or items being placed within the bag 100 and the hands of the non-sterile user.

As provided, the bag 100 may include a visible demarcation 108, such as, a solid line or other visible indication, located circumferentially around the protective cuff 106. The visible demarcation 108 visibly separates the designated non-sterile part of the bag 100 from the sterile part of the bag 100. Both parties (i.e., the scrubbed user and the non-scrubbed user) must understand which side of the visible demarcation 108 they are permitted to touch. The person with non-scrubbed hands may only touch the interior surfaces of the bag 100 and the exterior of the protective cuff 106 distal to the visible demarcation 108. The person with scrubbed hands may only touch the exterior of the bag 100 and the exterior of the protective cuff 106 proximal to the visible demarcation 108. As such, the visible demarcation 108 serves the purpose of removing any uncertainties about which surfaces of the bag 100 are being maintained sterile and which are not.

It is contemplated that in some embodiments, the visible demarcation 108 may be provided with adequate width to provide a buffer between the sterile and non-sterile zones, to help prevent any accidental overlap between the two zones. For example, the visible demarcation 108 may include two lines spaced apart a given distance, such as, for example, one inch. Alternatively, the visible demarcation 108 may be a cross-hatched, colored or otherwise identified buffer zone between the sterile and non-sterile zones.

It is further contemplated that the visible demarcation 108 may be overprinted onto a substrate such that when the bag 100 is configured to provide the protective cuff 106 the users may see the visible demarcation 108 and after the protective cuff 106 is involuted the users will see only the substrate, not the visible demarcation 108. This embodiment may help to reduce any confusion that may occur as to which side of the bag 100 one is viewing at any point in time.

After all of the non-sterile contents have been added to the bag 100, the protective cuff 106 may be involuted by the scrubbed person, by manipulating the protective cuff 106 from the proximal side of the visible demarcation 108, until the visible demarcation 108 (including the buffer zone, if present) is contained inside the bag 100. The bag 100 may be sealed proximal to the visible demarcation 108 by the closure mechanism (examples of which are provided herein with respect to FIGS. 3-6). The closure mechanism may be located inside or outside of the bag 100, but should be positioned in the sterile area (i.e., proximal to the visible demarcation 108) to assure that the non-sterile contents are securely contained enabling the secured bag 100 to be handled casually by a user with scrubbed hands.

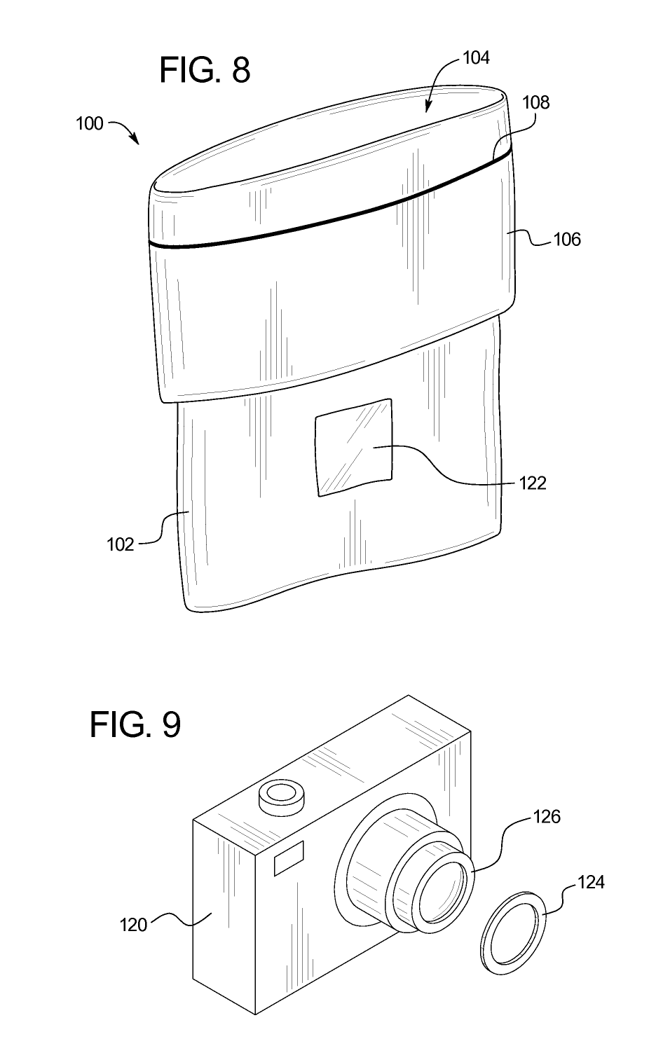

Turning now to FIG. 8, when used with a camera 120, or other optics-related electronic device, the bag 100 may contain an area, such as a lens window 122, of sufficient optical clarity to allow photos to be taken through the bag 100. This area could be the wall of the bag 100 itself, if a suitable material is used, or a discrete flexible or rigid lens window 122 through which the camera lens can see to take the picture. If a discrete window is used, the wall of the bag 100 may still be optically clear enough for the flash to be used through it or an additional flash window may be provided. The lens window 122 may function similar to a lens filter, providing protection to the lens, while allowing all lens functions (focusing, exposure, etc.) to operate normally.

The bag 100 may be provided with a mechanism that enables the surface of the lens to be mounted against the lens window 122. There are several advantages achieved by mounting the lens to the lens window 122. For example, the mechanism for mounting the lens to the lens window 122 may help to prevent wrinkles or other non-uniformities from forming in the lens window 122, whereas such non-uniformities may otherwise distort the image photographed through the lens window 122. Further, mounting the lens to the lens window 122 in a manner that isolates the lens from the remainder of the interior of the bag 100 may help to prevent stray internal reflections from the flash or other light sources from distorting or improperly exposing the photographic image. Moreover, mounting the lens to the lens window 122 may assist in keeping the camera 120 and bag 100 aligned with each other properly during use.

As shown in FIG. 9, in one contemplated embodiment of the present invention, the lens is secured to the lens window 122 using a double-sticky bezel ring 124 of material of appropriate inner and outer diameter to attach the lens bezel 126 to the lens window 122 area of the bag 100. Such an example may assisting in holding the lens window 122 taut across the lens opening, while isolating the lens from any internally reflected light from within the bag 100. In a preferred embodiment, the bezel ring 124 should be sufficiently adhesive to keep the lens bezel 126 in contact with the lens window 124, but not so adhesive as to prevent repositioning of the lens if it were initially positioned incorrectly or came loose during use. In addition, the bezel ring 124 may have different adhesives on each side to allow the ring to stay adhered to the lens bezel 126, but be repositioned against the lens window 122 during use. Further, it is understood that the adhesive on the lens bezel 126 side of the bezel ring 124 may need to allow for the eventual removal of the bezel ring 124 from the lens bezel 126 after use, without damaging or leaving residue on the lens bezel 126.

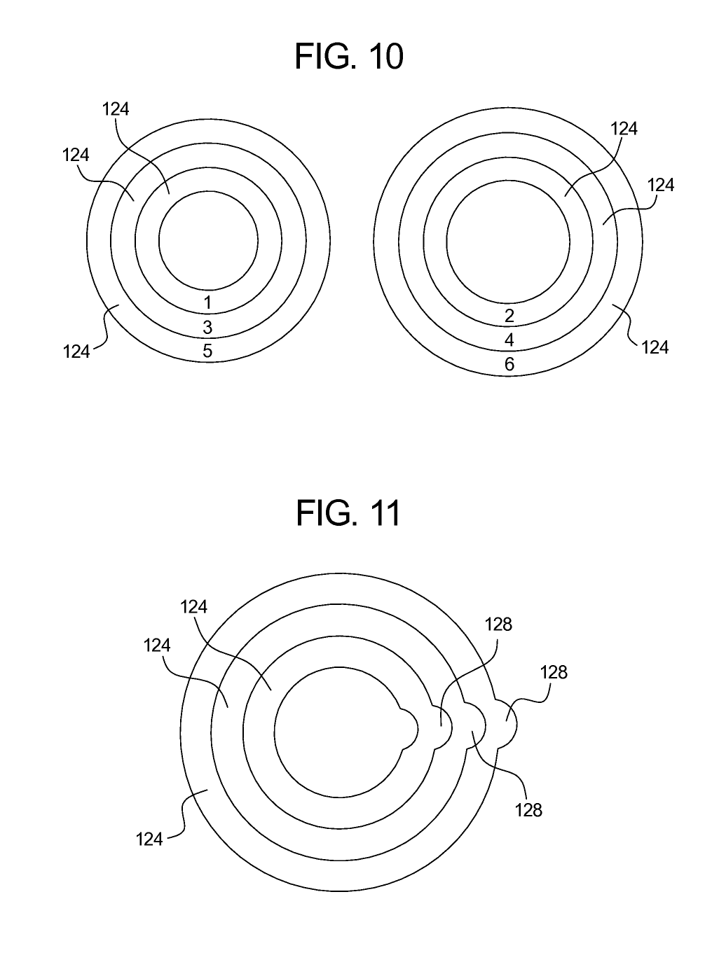

As shown in FIG. 10, to accommodate a variety of cameras 120, the bezel ring 124 may be provided in multiple sizes, formed out of die-cut material, with concentric bezel rings 124 placed together on the same release liner. The user may then select the correct size bezel ring 124 to use to appropriately match the camera 120. As shown, the size selection may be aided, for example, by a color, number, or letter coding scheme (e.g., the numbers 1, 2, 3, 4, 5 and 6 associated with each of the various sizes shown in FIG. 10). As further shown, if a single set of concentric bezel rings 124 may not be sufficient to cover all intermediate sizes, two or more sets of bezel rings 124 may be provided with each bag 100.

As shown in the example provided in FIG. 11, the bezel rings 124 may also include one or more die-cut tab 128 on each to allow easier removal from the release liner, application to the lens bezel 126, and ultimate removal from the lens bezel 126 after use. Multiple tabs 128 may be provided at various positions around the bezel rings 124.

While the adhesion of the lens bezel 126 to the lens window 122 has been described above with respect to using a bezel ring 124, it is understood that this is merely one embodiment of an adhesive mechanism that may be used to secure the lens bezel 126 to the lens window 122 or to the bag 100 itself. In other contemplated examples, the adhesive mechanism may be adhesive tabs, adhesive strips, adhesive bars or other sticky material or materials. For example, in addition to a plurality of bezel rings 124, a release liner may further include a series of die cut adhesive bars which may be used when none of the bezel rings 124 are appropriately sized.

In use, the non-scrubbed user (i.e., the “assistant”) removes the bezel ring 124 from its packaging, removes the release liner on the bezel side and applies the bezel ring 124 to the surface of the lens bezel 126, leaving the opposite side release liner in place. Then the assistant peels open the sterilization packaging from the bag 100 and presents the bag 100 to the scrubbed user according to standard aseptic presentation technique used with sterile packaging in the operating room. The scrubbed user then unfolds the bag 100 to open it, holding his or her fingers beneath the edges of the protective cuff 106 on the proximal side of the visible demarcation 108 to avoid contact with the camera 120 as it is placed into the bag 100. Next, the assistant removes the release liner from the outward-facing side of the bezel ring 124. He or she then takes the camera 120 and places it in the bag 100, aligning the bezel ring 124 with the lens window 122 area of the bag 100, if applicable.

The scrubbed user may help adhere the bezel ring 124 to the lens window 122, by pressing the outside sterile surface of the bag 100 against the lens bezel 126. If the lens needs to be repositioned, the assistant may remove the camera 120 from the bag 100, unsticking the bezel ring 124 from the lens window 122, and reposition the lens correctly.

When the non-sterile camera 120 is securely attached to the lens window 122 and contained completely inside the bag 100, the scrubbed user closes the bag 100, as described further above. The scrubbed user then proceeds to use the camera 120 normally, looking through the wall of the bag 100 to see the displays and controls of the camera 120, and manipulating the controls through the flexible wall of the bag 100.

While described above with reference to a bezel ring 124, it is understood that this embodiment may work best with cameras 120 that have flat lens bezels 126, perpendicular to the axis of the lens, and which do not rotate when the lens is extended or retracted. Many cameras 120 today have this feature, including many digital cameras 120. However, it is contemplated that there may any number of alternate methods and mechanisms available for attaching a lens bezel 126 to the lens window 122. For example, notable mechanical means of attachment such as rings, clips, etc. may be employed.

With respect to packaging the invention, the bag 100 may be packaged in a sterilization package and sterilized prior to opening and use. The bezel ring(s) 124 may be packaged separately from the bag 100 and need not be sterilized as it will be handled by the non-scrubbed user and will remain inside the bag 100 during use.

It is contemplated that embodiments of the sterile bag 100 and method for placing a non-sterile object within a sterile bag 100 may be executed without the need for a lens window 122. For example, in instances in which the packaging material is appropriately translucent or transparent to enable the satisfactory operation or manipulation of the materials within the bag 100, or in embodiments in which the opacity of the bag 100 is otherwise not an issue, the sterile bag 100 and closure may simply include a mechanism for sterile closure as described above with reference to FIGS. 1-7 without a need for the elements described with respect to FIGS. 8-11.

It should be noted that various changes and modifications to the presently preferred embodiments described herein will be apparent to those skilled in the art. Such changes and modifications may be made without departing from the spirit and scope of the present invention and without diminishing its attendant advantages.

I claim:

1. A method of using a non-sterile object in a sterile environment comprising the steps of:
providing a sterile bag including:
a body of the bag;
a sealed first end;
a second end that forms a cuff and an opening to an internal portion of the bag when in a first position;
a visible demarcation located circumferentially along the cuff dividing the bag into a proximal side towards the sealed first end and a distal side towards the internal portion of the bag;
a closure mechanism located along the cuff on the proximal side of the visible demarcation such that when the second end is in a second position there is no cuff and the closure mechanism can be used to seal the bag with the visible demarcation sealed within the bag;
a first user having scrubbed hands grasping the bag proximal to the visible demarcation such that the first user's hands are partially covered by the cuff;
a second user inserting a non-sterile object into the bag without contacting the bag other than distal to the visible demarcation;
the first user manipulating the bag and closure mechanism to seal the non-sterile object and every portion of the bag distal to the visible demarcation within the sealed portion of the bag, without touching the bag anywhere proximal to the visible demarcation.

2. The method of claim 1 wherein the non-sterile object is a camera, the bag further includes a lens window and further including the steps of:
providing at least one bezel ring;
the second user attaching one side of the bezel ring to the bezel of the camera and the other side of the bezel ring to the lens window before the first user seals the camera within the bag.

3. The method of claim 2 wherein the bezel ring is an adhesive bezel ring.

4. The method of claim 2 wherein the step of providing at least one bezel ring includes providing a plurality of sizes of bezel rings from which the lens bezel ring may be selected to match the bezel of the camera.
